(19) United States
(12) Patent Application Publication
(10) Pub. No.: US 2014/0107741 A1
(43) Pub. Date: Apr. 17, 2014

(54) Neurostimulator Lead Extraction Tool

(71) Applicant: Dennis Hyun Kim, Los Angeles, CA (US)

(72) Inventor: Dennis Hyun Kim, Los Angeles, CA (US)

(21) Appl. No.: 13/650,319

(22) Filed: Oct. 12, 2012

(51) Int. Cl. A61N 1/05

(52) U.S. Cl. CPC A61N 1/0551
USPC 607/116

(57) ABSTRACT

The device relates to a neurostimulator lead extraction tool, which aids in the safe and minimally invasive extraction of an anchored neurostimulator lead. The device is characterized by a handle, barrel, and cutting surface, in that the inner surface of the barrel is advanced over the outer surface of a neurostimulator lead thereby guiding the cutting barrel of the tool through body tissues until the cutting surface is against anti-migration tines of the neurostimulator lead. The device is rotated axially in order to engage the cutting surface. As each set of tines is cut, the cutting surface can be advanced up to the next tine until all the tines are severed. The device and the neurostimulator lead are then removed along the same path of entry, preventing the cutting tool and the lead from migrating into surrounding nerves, blood vessels, and organs thereby avoiding damage to the same.

### TECHNICAL FIELD

The device relates to the surgical removal of a neurostimulator lead and more particularly to instruments that aid in safe and minimally invasive extraction of an anchored neurostimulator lead in patients having undergone procedures relating to neuromodulation implants.

### BACKGROUND

Sacral neuromodulation is a medical treatment utilizing a surgical implant device indicated in the treatment of medically refractory urinary frequency and urgency with or without urinary incontinence as well as idiopathic (non-obstructive) urinary retention. Recently in the US, it was also FDA approved for the treatment of fecal incontinence. Other disease conditions that are currently being investigated worldwide as possible indications include: interstitial cystitis, chronic pelvic pain, and chronic constipation. As a whole, the number of implants done worldwide continues to increase. To date over 100,000 implants have been performed worldwide.

A certain percentage of implants eventually require removal for various reasons such as lack of efficacy, patient choice, implant pain, infection, device malfunction, lead migration, or necessity to have an MRI. Currently, the need to have an MRI is a contraindication for receiving a sacral neuromodulation implant. This is due to concerns that the radiofrequency energy generated by the magnetic field can cause the electrode to heat and potentially cause tissue and nerve damage.

The sacral neuromodulation implant can be thought of as having two parts: an internal pulse generator and a neurostimulator lead. The lead on one end has a series of metal electrodes designed to lie near nerves and stimulate them, followed by several rows of small plastic barbs or tines as sometimes referred, to prevent the lead from migrating, and on the other end, a series of lead connectors that attach to the internal pulse generator. The electrode end of the lead is placed through the skin of the upper buttock and through a sacral foramen, which is one of several small openings in the sacral bone to its desired position near the underlying nerves to stimulate the same and exert its effects. The internal pulse generator is then attached to the connection end of the lead and implanted into a subcutaneous pocket in the upper buttock of the patient.

One of the earlier problems with lead placement was that the lead had a tendency to migrate out of its desired position. The sacral neuromodulation implant was thus modified to include a tined lead, a series of small plastic barbs that served to anchor the lead into proper position and prevent lead migration. While the tined lead has been an improvement with respect to the problem of lead migration, it has made the complete removal of the lead difficult and unpredictable.

Complete lead removal is additionally difficult in that the tines and the lead electrodes lie underneath the sacral bone which prevents easy surgical access. The lead when properly placed traverses the sacrum through a sacral foramen. Within the sacrum run several nerves controlling motor and sensory function to the genitalia, pelvic floor musculature, rectum and bladder. These nerves are vital in the normal function of these structures. Beneath the sacrum also lie the main trunks of several large nerves and blood vessels that supply the legs. Complete lead removal can put these important structures in an area of densely packed anatomy at risk of serious permanent injury.

Currently the technique of sacral neuromodulation implant removal can be broken down into two parts: the removal of the internal pulse generator and the neurostimulator lead. The internal pulse generator is removed by making an incision in the skin overlying the generator through the previous incision and simply retrieving the device. The connection end of the neurostimulator lead that attaches to the internal pulse generator is then detached and retrieved by bringing it out through a separate small skin incision over the sacrum. Most if not all experts “remove” the lead by applying gentle traction until the lead either pulls free, if the lead has not been in place for longer than a few weeks, but more commonly the tines prevent removal and the end of the lead breaks off leaving the tines and metal electrodes behind in the body beneath the sacrum. This can lead to persistent pain and discomfort. A further disadvantage of the current removal procedure can lead to the retained fragments migrating further into the body and requiring additional surgery at a more invasive level further increasing the risk of permanent serious injury. Finally, the fragments continue to pose a risk for those requiring any MRI procedures in that the remaining section of neurostimulator lead is subject to the radiofrequency energy generated by the magnetic field and can heat up causing potential internal tissue damage. As such there is a need for a device to reliably and safely remove the neurostimulator lead in a minimally invasive way.

A prior art search for comparable devices yielded a few devices with some similarities yet fundamental differences such that the proposed neurostimulator lead extraction tool is materially different in form and its unique function. It should be noted for the avoidance of doubt that anything stated or otherwise disclosed within this application is not admitted prior art expressly or impliedly.

In reference to O'Neill U.S. Pat. No. RE40,796E, a tool for harvesting bone: This tool is essentially a coring device with a cutting drill head. The guidance mechanism of the bone harvesting tool is different in that it involves manually placing a guidewire into the target tissue, using a series of obdurators and sheaths to create a working space and then using a drill and coring device within the sheath to remove a plug of tissue. The dilating obdurator and sheath system would be wholly unsuited for the removal of the neurotransmitter lead. The O'Neill tool would not be minimally invasive as the required working channel would be larger than necessary, likely cause more bleeding, and based upon the anatomy of the sacrum would not physically fit through the sacral foramina. Finally the coring device and cutting drill head is such that it is compatible only with an outer working sheath and would not be compatible with the deployment over a guiding wire. As such it would sever the wire and render its design as a neurostimulator lead removal device worthless.

In reference to Collinsworth U.S. Pat. No. 5,895,403, which is a tool for cutting and extracting hair plugs: This tool consists of a cylindrical stepped diameter chamber with a cutting surface on the end of the cylinder. As the cutting end is pressed into the scalp a plug of tissue containing hair follicles is partitioned. Vacuum suction is then applied and by way of the variable diameter chamber airflow is allowed past the tissue plug suctioning it free. This tool by way of its design requires an ever-widening caliber to accommodate the flow of air. This type of design would severely limit its utility as a neurostimulator lead removal tool by virtue of the tool's outer diameter becoming progressively wider. By becoming wider the tool would become progressively more difficult to pass through deeper layers of tissue. Additional downward force would then be required to pass the tool deeper. But this additional force can lead to sudden slippage and lack of control over depth of penetration therefore making the use of this tool prohibitively dangerous. The thicker wall would also result in a greater surface area on aspect and tend to catch its edge as it is passed through the small tight spaces in between fixed bony structures thus limiting its ability to get the cutting surface to the desired location of the lead. Finally the use of vacuum suction for lead removal would be inefficient, unreliable and wholly unnecessary.

In reference to Lewis U.S. Pat. No. 7,367,804 B2, an extractor tool and method to retrieve a broken endodontic drill bit. This tool primarily encompasses an extractor tool that upon heating, grips a broken metal drill bit to allow for its extraction. A secondary tool is a hollow bore coring tool to remove surrounding bony enamel from around the end of the broken drill bit. This particular coring tool would be inappropriate to use in removal of a neurostimulator lead. This design would not be able to take advantage of the existing neurostimulator lead to act as its own guide and safety wire to allow proper positioning of the cutting surface onto and only onto the targeted tines for removal. Without this mechanism the core of tissue within the tool needs to be constantly cleared to allow further passage of the tool into deeper layers. By sliding the tool over the lead in addition to proper targeting and safety the problem of having to remove tissue within its core is eliminated as there is no tissue only the preexisting lead. Furthermore, from the illustrated example embodiments, the thickness of the coring tool walls appear to be prohibitively too thick rendering the passage of such a tool through tissue unnecessarily difficult. As the wall thickness of the tool increases, the probability of the tool being able to navigate the small interstices between bony structures to reach its target for cutting decrease. Lastly the coring tool is just a coring tool only and has no function as an extractor.

In reference to Fischell U.S. Pat. No. 4,898,575, a guidewire tunneling catheter system for removing atherosclerotic plaque. In its essence this tool utilizes a guidewire system to properly position a cutting blade against atherosclerotic plaque to aid in its safe removal. This tool however requires the use of an outer working sheath to guide placement of the guidewire and boring tool. The use of an outer sheath in this manner would render the attempted removal of a neurostimulator lead ineffective. The outer sheath in practice would only work in a fluid environment where advancement of the sheath could still allow function of the inner chamber parts by means of simply displacing or suctioning off the excess fluid. Trying to utilize such a tool through solid tissue would physically create a plug of cored tissue that would need to be extracted from within the sheath as the sheath was advanced. Additionally, removal of a neurostimulator lead by virtue of its close quarters with other structures including protective bone would preclude passage of instruments that may enlarge the passageway already inhabited by the lead itself. As such, the proposed device would fit precisely over the outer diameter of the lead so as to slide directly over the lead itself, minimizing any potential tissue drag, not trapping a core of tissue beneath it that would obstruct its advancement, and better allow it to fit in between small openings in bone. The Fischell tool also would not be able to adjust to the variability between the relatively short fixed lead and its relation to the various depths within the body that the tines lay. The present device was designed to uniquely conform to a neurostimulator lead and its possible relationships to the body and the anchoring sites of the lead within.

### SUMMARY

Thus the present device was conceived to not only remove the neurostimulator lead, but do so in such a way as to utilize the lead itself as a guide to properly and safely position the cutting surface to release the lead from its anchoring tines and allow the device to progress through body tissues in a minimally invasive manner. Utilizing the lead itself as a guide ensures that the cutting surface always maintains close safe contact to the lead and inhibits the cutting surface of the tool from cutting any surrounding structures. In addition to acting as its own guidance system and cutting instrument, the tool finally serves to function as its own extractor.

Another object of the device is to utilize real time intra-operative fluoroscopic X-ray imaging to help visualize and localize its precise anatomical location during the delicate lead extraction process.

Patients who require neurostimulator lead removal come in various body shapes and sizes with respect to their body fat composition and hence body wall thickness. As such, the depth of the anchoring tines relative to the skin can be highly variable whereas the length of the undamaged lead is fixed. In those patients who have a lead that is anchored deeply or in those where the lead may have been cut short, a problem may arise where the tool may actually be longer than the lead available for manipulation above the skin. In this situation, if the tool were passed over the lead, the free end of the lead would not extend beyond the length of the tool, and the tool could not be safely deployed over the lead as a guide as some degree of back tension is required on the lead such that the lead does not buckle resulting in the cutting surface either transecting the lead rather than shearing off the tines and/or inadvertently damaging surrounding tissue, Additionally, even if the tines were successfully cut from the lead, without the back tension, unnecessary cutting may continue even though the lead is free and available for removal thus making the lead removal dangerous.

To account for these contingencies with respect to the present device versus lead proportions, it is another object of the device that the handle is designed to manage these situations. Finally by way of this design, the manufacture of this tool is simplified into a “one size fits all” thereby saving manufacturing and design costs.

This particular device aids in the complete removal of the neurostimulator lead without the end of the lead breaking off and being left within the body of the patient. The device can accomplish this objective reliably, safely, and minimally invasively without the need of a larger more morbid skin incision, exposure of the sacrum or removal of bone, or serious injury to major surrounding nerves, blood vessels, or pelvic organs.

### BRIEF DESCRIPTION OF DRAWINGS

Example embodiments of the device are described below with reference to the following accompanying drawings.

FIG. 1 shows the side view of an embodiment of the device;

FIG. 2 shows a magnified front view of the handle and end of the barrel housed within;

FIG. 3 shows a magnified side view of the cutting end of the barrel;

FIG. 4 shows an end on view of the cutting end of the barrel;

FIG. 5 shows a view of the tool being deployed over the lead as well as the relationship of the target lead tines and electrodes buried within the sacral bone directly adjacent to major nerves, blood vessels, and pelvic organs;

FIG. 6 is a magnified view of the cutting end of the tool approaching the neurostimulator lead tines;

FIG. 7 is a magnified view of the cutting end of the device being rotated to cut the tines free;

FIG. 8 is a magnified view of the cutting end of the tool advancing further along the lead to release the next set of tines;

FIG. 9 shows the device position after fully advancing to transect all the anchoring lead tines and the lead now free for removal.

### DETAILED DESCRIPTION OF DRAWINGS

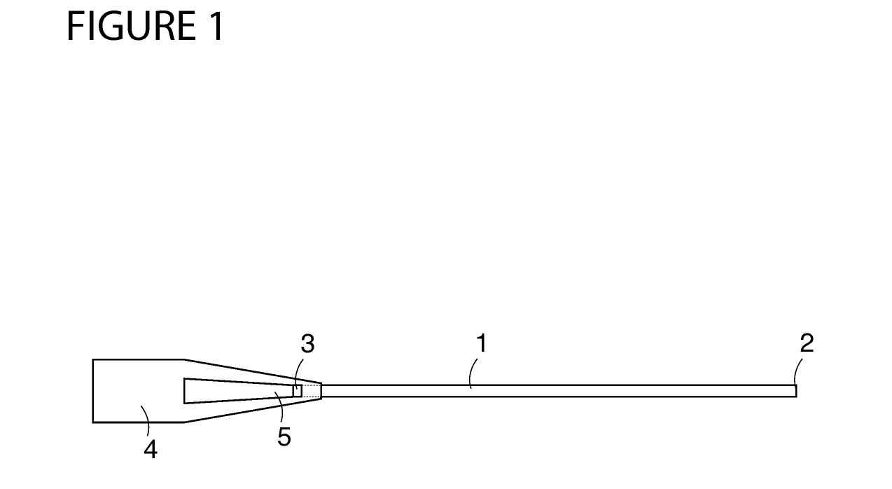

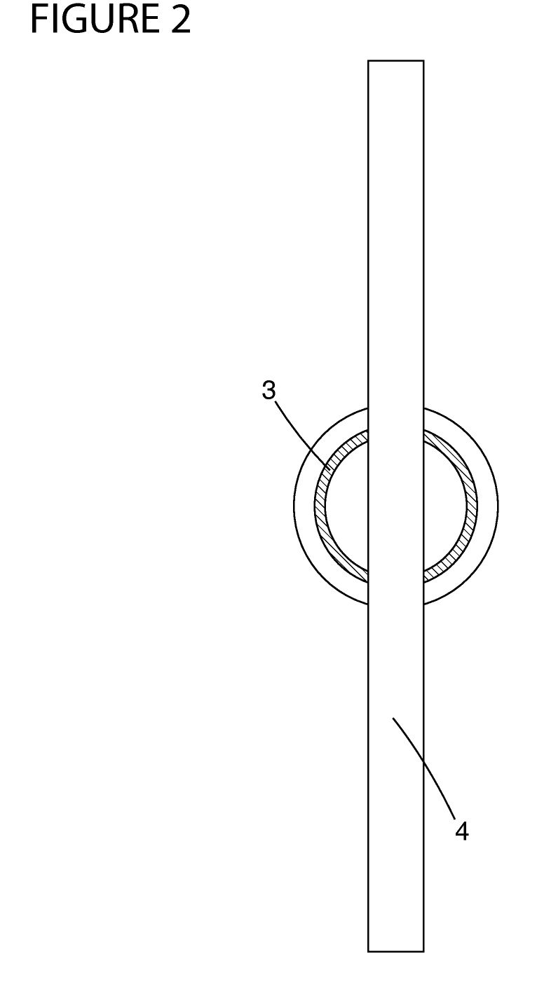

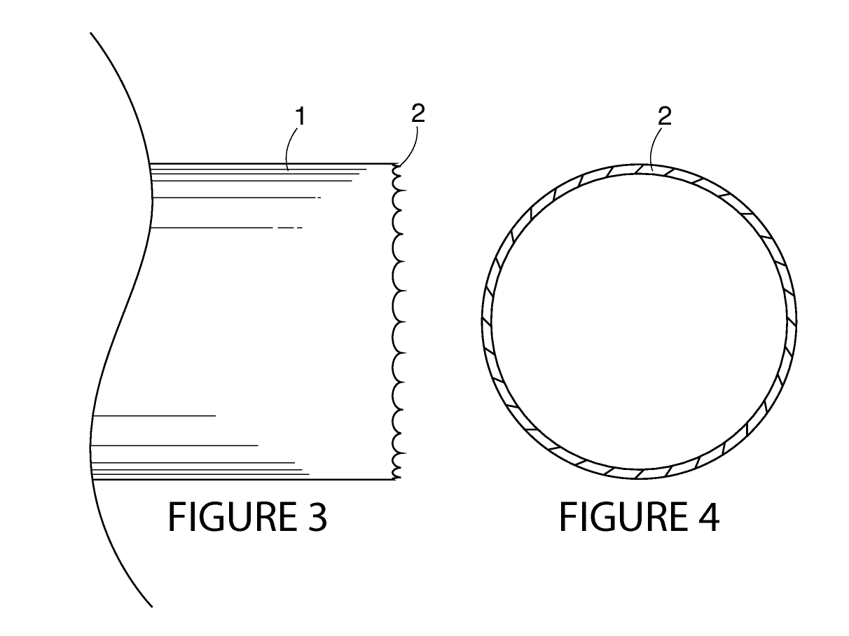

Referring to FIGS. 1, 2, 3 and 4, an embodiment of the neurostimulator lead removal device is shown as having a barrel 1 with an inner lumen diameter greater than the outer diameter of the neurotransmitter lead, wherein at one end of said barrel there is a cutting surface 2, and at the other end of said barrel 3 there is a handle 4, which is fused or otherwise attached to the barrel.

In accordance with a further embodiment of the device in FIG. 1, the barrel diameter is greater than the outer diameter of the core section of the tines 13 such that the amount of tine material that requires cutting is minimized.

In accordance with a further embodiment of the device in FIG. 1, the barrel 1 is between 3.5 inches to 5.5 inches in length made of surgical steel or similar material having an inner lumen that is 0.066 inches in diameter and is radio-opaque. The handle is an additional 1.5 to 2.5 inches in length from the point of attachment. The end of the barrel 3, attached to the handle, terminates at the point of attachment to the handle or shortly thereafter. Contiguous with the attached end of the barrel is an opening 5, within the handle 4. The opening is wide enough to allow the neurostimulator lead to exit away from the handle. This allows early exposure of the lead to help maintain back-tension on the lead, thereby helping to prevent transecting the lead, ease guidance of the cutting end of the barrel, and ensure safe operation of the device.

In another embodiment of the device the handle 4 is tapered or otherwise of varying width beginning at the free end of the handle where it is of a larger dimensions to that of the handle at the point of attachment at the attaching end of the barrel 3 where the width of the handle is nearly the same as the outer diameter of the barrel. In this embodiment of the device the tapered design of the handle allows the handle to be inserted into a skin incision to enable the cutting surface of the barrel 2 to reach deeper tissue without the need of a larger more morbid skin incision.

In another embodiment of the device of FIG. 1, the cutting surface 2 is a bi-directional serrated edge.

In another embodiment of the device of FIG. 1, the barrel is made of surgical grade steel or any material acceptable for use in performing surgical procedures, where the barrel can be formed by, for example but not limited to, extrusion, forging, stamping, rolling, or die casting. The handle can be made of, for example but not limited to, metal, plastic, rubber, PVC, or any material suited to providing a solid gripping surface and capable of being positively attached to the barrel by, for example but not limited to, adhesives, welding, friction welding, thermal bonding, or any other method by which the handle can be fused or otherwise positively attached to the barrel.

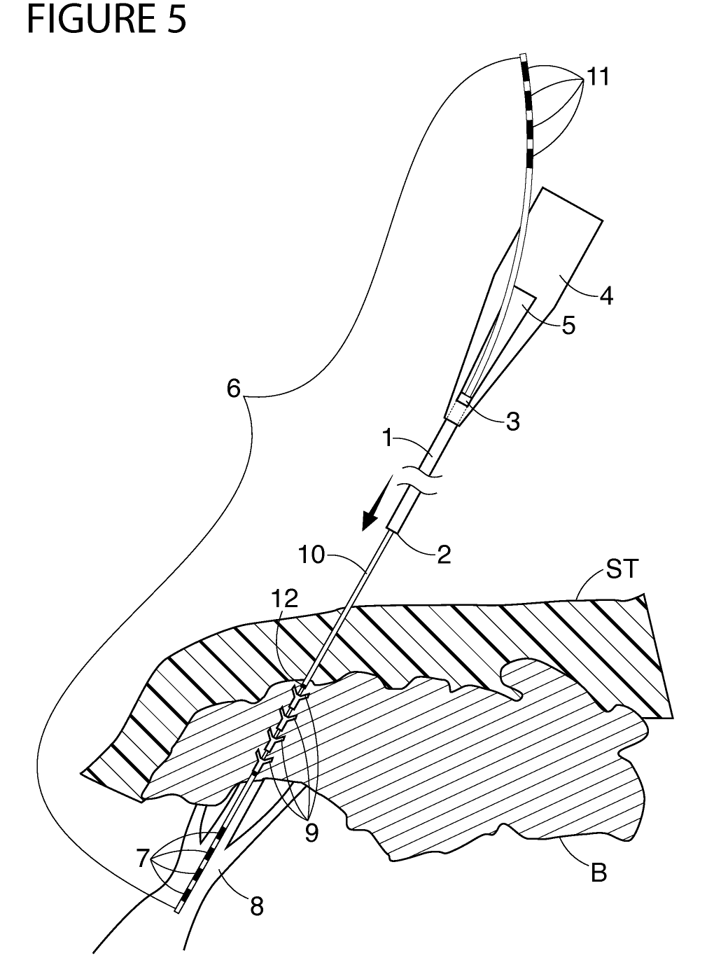

FIG. 5 shows a neurostimulator lead 6. On the distal end of the lead are four electrodes 7 that lie adjacent to the pelvic nerves 8 and are responsible for exerting the effects of the neurostimulation. More proximally there are four tines 9. Proximally still is the body of the lead 10, and at its most proximal end 11, four lead connector sites that attach to the neurostimulator generator (not shown). The barrel 1 with its cutting surface 2 is shown being threaded over the neurostimulator lead 6. The proximal end of the lead with lead connector sites 11 is seen exiting through the proximal end of the barrel 3 (also referred to as the attached end of the barrel or the end attached to the handle), and out of the slot 5 in the handle 4.

In this position the cutting surface of the barrel 2 is being guided over the body of the lead 10 and is either still above the level of the patient's skin or still in the superficial layers of tissue and not near any deeper pelvic structures. At this point the proximal lead end 11 is grasped with a clamp and gentle back tension is applied thereby introducing tension on the lead. This helps straighten the lead and maintain it in a straighter configuration as the tool is advanced over the lead so the lead will not buckle. This ensures that the cutting surface 2 is maintained in a safe position sliding over the body of the lead 10 at all times.

Just proximal to the tines 9, is a radio-opaque marker 12 that provides a radiographic landmark as to the depth of the tines 9 when obtaining real time X-ray imaging of the patient. This marker 12 can be viewed in relation to the cutting surface of the barrel 2 when the tool is being advanced over the lead.

Eventually with constant back tension, the cutting surface 2 will encounter resistance as it runs into the lead tines 9. Proper tool position can further be confirmed with the use of real time fluoroscopic X-ray imaging as the relationship between the tine radio-opaque marker 12 can easily be seen with respect to the leading cutting surface of the barrel 2.

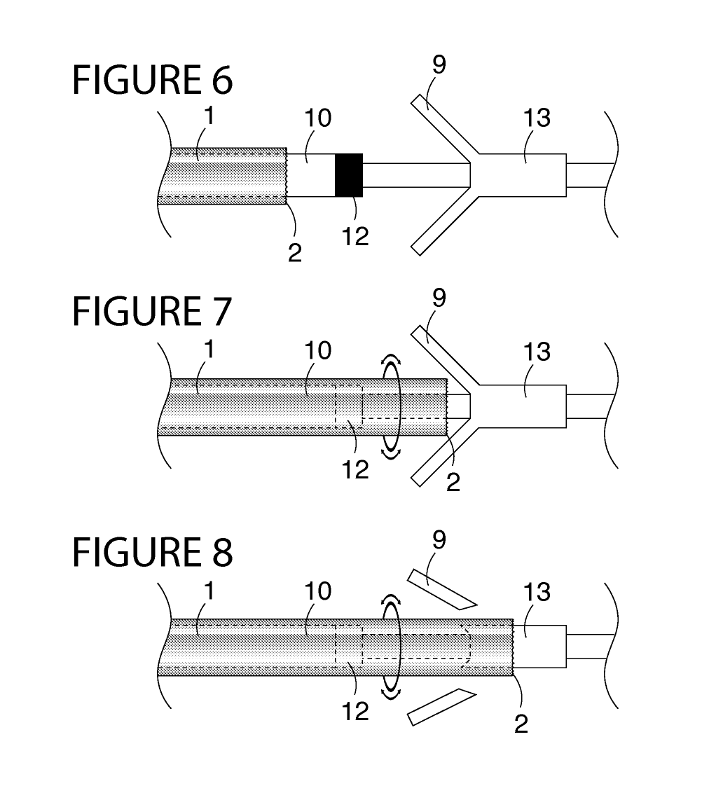

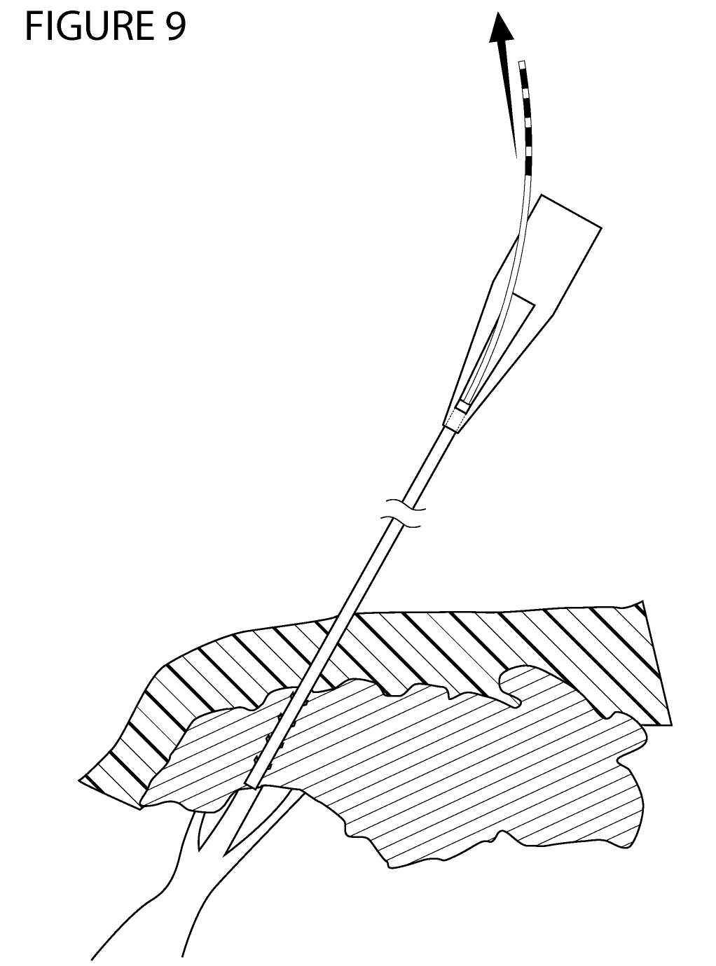

FIGS. 6, 7, and 8 depict a magnified view of the tine cutting process. Once the cutting surface has encountered resistance and proper position is confirmed with X-ray imaging, a gentle twisting motion is applied to the handle to rotate the tool in a continuous twisting fashion, or in a back and forth motion. The cutting surface will then cut the plastic tines 9 free of the lead body 10. As each row of tines is released, the tool will be able to advance a small distance until the next row of tines is encountered repeating the cutting process until all the tines are cut which again can be confirmed on X-ray imaging.

FIG. 9 shows successful release of the anchoring tines from the lead. The lead is now free and easily removed intact along with the barrel 1.

Various other modifications, adaptations and alternative designs are of course possible in light of the above teachings. Therefore, it should be understood at this time that within the scope of the appended claims, the device may be practiced otherwise than as specifically described therein.

The device claimed is:

1. A neurostimulator lead extraction device comprising:
a hollow barrel adapted for encircling a neurostimulator lead;
a handle attached to one end of the barrel; and
a cutting surface at the opposite end of the barrel.

2. The device of claim 1, wherein said barrel is cylindrical.

3. The device of claim 1, wherein said handle has an opening through which there is access to the inner diameter of the barrel.

4. The device of claim 3, wherein the handle is tapered such that the dimensions of the handle at its free end are greater than its dimensions at the point of attachment to the barrel.

5. The device of claim 4, wherein the thickness of the handle is less than the diameter of the barrel and the width of the handle at the free end is less than half the length of the handle.

6. The device of claim 1, wherein the barrel is made of a radio-opaque material.

7. A neurostimulator lead extraction device comprising:
a hollow barrel adapted for encircling a neurostimulator lead having two ends, an attaching end, and an opposite end, wherein, the inner lumen diameter of the barrel is greater than the outer diameter of a neurotransmitter lead;
a handle, which is fused or otherwise attached to the attaching end of the barrel;
a cutting surface at the opposite end of said barrel.

8. The device of claim 7, wherein the barrel is cylindrical.

9. The device of claim 7, wherein the handle has an opening through which there is access to the inner diameter of the barrel.

10. The device of claim 9, wherein the handle is tapered such that its dimensions at its free end are greater than its dimensions at the point of attachment to the barrel.

11. The device of claim 10, wherein the thickness of the handle is less than the diameter of the barrel and the width of the handle at the free end is less than half the length of the handle.

12. The device of claim 11, wherein the barrel is made of a radio-opaque material.

13. The device of claim 7 wherein the cutting surface is a bi-directional serrated cutting edge.

14. The device of claim 7, wherein the barrel is between 3.5 inches and 5.5 inches in length, made of surgical steel having an inner lumen that is 0.050 to 0.080 inches in diameter, is preferably radio-opaque, and said barrel terminates at or near the attachment point of the handle, wherein said handle is an additional 1.5 inches to 2.5 inches in length from the point of attachment to the barrel, wherein said handle has an opening contiguous with the attachment end of the barrel wherein said opening is wide enough to allow the neurostimulator lead to exit away from the handle.
